(19) United States
(12) Patent Application Publication
(10) Pub. No.: US 2004/0091951 A1
(43) Pub. Date: May 13, 2004

(54) Assay for measuring acetylation or deacetylation activity of an enzyme

(75) Inventor: Brian E. Schultz, Burlingame, CA (US)

Correspondence Address: Wayne W. Montgomery, AXYS PHARMACEUTICALS, INC., Vice President, Intellectual Property, 180 Kimball Way, So. San Francisco, CA, 94080, US

(73) Assignee: AXYS PHARMACEUTICALS, INC., So. San Francisco, CA (US)

(21) Appl. No.: 10/360,534

(22) Filed: Feb. 7, 2003

Related U.S. Application Data
(60) Provisional application No. 60/355,700, filed on Feb. 7, 2002.

(51) Int. Cl. G01N 33/53; G01N 33/537; G01N 33/543

(52) U.S. Cl. 435/7.92

(57) ABSTRACT

This invention is directed to a continuous method for measuring the activity of an enzyme that catalyzes the addition of an acetyl group to a residue capable of being acetylated or an enzyme that catalyzes the removal of an acetyl group from an acetylated residue. In particular the present invention is directed to a continuous method for measuring the activity of histone acetyltranferases and histone deacetylase enzymes.

### CROSS-REFERENCE

This application claims priority under 35 USC 119(e) to U.S. Provisional Application Serial No. 60/355,700, filed on Feb. 7, 2002, the disclosure of which is incorporated herein by reference in its entirety.

### FIELD OF THE INVENTION

This invention is directed to a continuous method for measuring the activity of an enzyme that catalyzes the addition of an acetyl group to a residue capable of being acetylated or an enzyme that catalyzes the removal of an acetyl group from an acetylated residue. In particular the present invention is directed to a continuous method for measuring the activity of histone acetyltranferases and histone deacetylase enzymes.

### STATE OF THE ART

Acetylation and deacetylation of histone proteins, transcription factors, and related proteins play a major role in the control of cellular processes. In particular, the acetylation state of histones controls how tightly the histone proteins interact with DNA, and therefore how accessible the DNA is to transcription factors. Enzymes that add acetyl groups to histones or other proteins are called histone acetyltransferases (HATs). Enzymes that remove the acetyl groups fall into two families: the histone deacetylases (HDACs) and the Sir2 family of deacetylases. Currently there are eleven known members of the mammalian HDAC family (Gray and Ekström, Exper. Cell Res. 2001, 262, 75-83; Zhou, et al. Proc. Natl. Acad. Sci. USA 2001, 98, 10572-10577; Kao et al. J. Biol. Chem. 2002, 277, 187-193; Gao et al. J. Biol. Chem. 2002, 277, 25748-25755) and seven members of the Sir2 family (Gray and Ekström, Exper. Cell Res. 2001, 262, 75-83).

Histone acetyltransferases catalyze the transfer of an acetyl group from acetyl-CoA to the ε-amino group of a lysine residue on the target protein. Many HAT enzymes have been characterized from eukaryotic organisms (Sterner and Berger, Microbiol. Mol. Biol. Rev. 2000, 64, 435-459). HDAC enzymes utilize a zinc ion at the active site of the protein to catalyze the removal of the acetyl group from acetyllysine in the form of acetate. Members of the Sir2 family of enzymes use NAD as a cofactor in the hydrolysis of acetyllysine.

The acetylation state of histone proteins plays a major role in gene expression and in cell-cycle control, and appears to play a role in certain forms of cancer. In particular, abnormal recruitment of histone deacetylases by corepressor proteins has been shown to promote the development of promyelocytic leukemia. In tumor cell lines, several studies have shown that treatment with HDAC inhibitors can lead to growth inhibition, growth arrest, terminal differentiation, and/or apoptosis. In vivo studies have demonstrated growth inhibition of tumors and a reduction in tumor metastasis as a result of treatment with HDAC inhibitors (Krämer et al. Trends Endocrinol. Metab. 2001, 12, 294-300).

Effective study of the enzymology and inhibition of HATs, HDACs, and Sir2 enzymes depends on the availability of robust assays capable of being performed in a high-throughput manner. Several assay methodologies have been developed for these enzymes, with varying degrees of utility for inhibitor screening.

Histone acetyltransferase assays are typically radioactivity-based. In these formats, acetyl-CoA radiolabeled on the acetyl group is reacted with a peptide corresponding to a histone amino acid sequence. Transfer of radiolabeled acetate to the peptide is quantitated by binding of the peptide to affinity resin (Ait-Si-Ali et al. Nucleic Acids Res. 1998, 26, 3869-3870), phosphocellulose paper (Tanner et al. J. Biol. Chem. 1999, 274, 18157-18160), or scintillation microplates (Wynne Aherne et al. Methods 2002, 26, 245-53) and measurement of the associated radioactivity. In a non-radioactive coupled assay format, the free CoA formed in the acetyltransferase reaction serves as a susbstrate for α-ketoglutarate dehydrogenase or pyruvate dehydrogenase. Formation of NADH serves as a measure of the rate of acetyltransferase activity (Kim et al. Anal. Biochem. 2000, 280, 308-314).

The most common deacetylase assay methodology involves labeling lysine groups in histone peptides with radiolabeled acetate. The deacetylase enzyme removes the acetyl group as acetate, which is subsequently isolated by extraction and quantitated on the basis of its radioactivity (Inoue and Fujimoto, Biochim. Biophys. Acta 1970, 220, 307-316). In a variant of this approach, a scintillation proximity assay, peptides derivatized with radiolabeled acetyl groups are attached to a bead containing scintillant that emits light upon exposure to radiation. In this assay format, cleavage of the acetyl groups causes a decrease in the light emission from the scintillant (Nare, et al., Anal. Biochem. 1999, 267, 390-396). A non-radioactivity-based assay uses peptides containing an acetyllysine group and a fluorescent tag. Reactivity is measured by high-performance liquid chromatography, using the difference in retention time of the acetylated and non-acetylated peptides to isolate and quantitate the reaction products (Hoffmann et al. Nucleic Acids Res. 1999, 27, 2057-8; Hoffmann et al. Bioconjug Chem. 2001,12, 51-5; Hoffmann et al. Arch Pharm (Weinheim) 2001, 334, 248-52). A commercial assay uses a two-step detection protocol. In the first step, a peptide containing an acetyllysine is reacted with a deacetylase for a given period of time. Following this, the reaction is quenched and the exposed lysine is reacted with a developing agent that produces a fluorophore, and the amount of deacetylated lysine is quantitated using the fluorescence of the product (Biomol, Plymouth Meeting, Pa., USA). More recently, a two-step, protease-coupled assay was reported, in which a peptide was designed containing a fluorescence resonance energy transfer (FRET) donor-quencher pair and an acetyllysine. After the deacetylase reaction has been allowed to run, the reaction is quenched and the amount of of deacetylated peptide is quantitated by reaction of the deacetylated peptide with a protease enzyme that cleaves specifically after lysine residues (Frey et al. Presented at 224th National Meeting of the American Chemical Society, Boston, Mass., August 2002; paper MEDI-121). To date, no continuous, non-radioactive histone deacetylase assays have been reported.

Features of the above assay formats limit their utility. Assays based on radioactivity tend to be costly, and require special handling precautions. Also, they are often difficult to perform in a high-throughput manner. Assays that measure activity on the basis of the disappearance of a signal with time rather that the appearance of signal usually yield poor signal/noise and signal/background ratios. Further, with the exception of the scintillation proximity assay described above, all deacetylase assays reported to date are endpoint assays, where the extent of the reaction is determined at a single time point only. The endpoint format assumes a linear rate of product formation with time. However, this assumption is not valid if the enzyme follows burst-phase or lag-phase kinetics, if the enzyme activity decreases over the course of the reaction, if the substrate is significantly depleted over the course of the reaction, or if substrate or protein aggregation occurs over the course of the reaction. A continous format, in which the progress of the reaction is monitored as the reaction proceeds, is more robust than an endpoint format.

Coupled assays are common in the practice of enzymology. The technique has been reviewed in the literature (Rudolph, et al. Methods Enz. 1979, 63, 22-42). In this type of assay, the formation of the product of an enzymatic reaction is not measured directly. Rather, the product reacts further with another enzyme or chemical to form a second product that has is readily detectable using spectroscopic or other detection methodologies. Representative examples of this methodology are a horseradish peroxidase-coupled assay for L-amino acid oxidase (Ueda et al. Toxicon 1988, 26, 695-706), a chymotrypsin-coupled assay for prolyl isomerase (Fischer et al. Biomed. Biochim. Acta 1984, 43, 1101-11), and an assay for methionine aminopeptidase in which the cleavage of a thioester bond in the enzyme substrate yields a free thiol that can react with 5,5′-dithio-bis(2-nitrobenzoic acid) (DTNB) to yield a detectable chromophore (Zhou et al. Anal. Biochem. 2000, 280, 159-165). In a coupled enzyme system, if the second reaction is slower than the first, an endpoint format is necessary. The first reaction must be quenched, and the second reaction is used to quantify the amount of product formed. In the case where the second reaction is more rapid, both reactions can be run at the same time. A steady-state can be achieved in which the rate of production of the first product will equal the rate of its conversion into the second product. Under this condition, the signal produced by the formation of the second product will be a measure of the formation of the first product, and thus a measure of the activity of the first enzyme. This allows for a continuous assay format, in which the enzymatic activity can be monitored directly as a function of time.

### SUMMARY OF THE INVENTION

In one aspect, this invention is directed to a continuous method for measuring the activity of an enzyme that catalyzes (a) the addition of an acetyl group to a residue capable of being acetylated or (b) removal of an acetyl group from an acetylated residue which method comprises incubating said enzyme with:

(i) a protease;

(ii) a polypeptide comprising:

(a) a recognition site for the protease;

(b) a residue, in which the acetylation state of the residue modifies the rate of cleavage of the polypeptide by the protease; and

(c) at least one chemical moiety, attached to the polypeptide, that possesses an optical signal that changes upon cleavage of the polypeptide; and

(iii) measuring the change in the optical signal.

In a second aspect, this invention is directed to a continuous method for measuring the activity of an enzyme that catalyzes the addition of an acetyl group to a lysine residue or an enzyme that catalyzes the removal of acetyl group from N^{ε}-acetylated lysine residue which method comprises incubating said enzyme with:

(i) a protease;

(ii) a polypeptide comprising:

(a) a recognition site for the protease;

(b) a lysine or acetyllysine residue, in which the acetylation state of the residue modifies the rate of cleavage of the polypeptide by the protease; and

(c) at least one chemical moiety, attached to the polypeptide, that possesses an optical signal that changes upon cleavage of the polypeptide; and

(iii) measuring the change in the optical signal.

Preferably, the method measures the activity of an enzyme that catalyzes the removal of acetyl group from N^{ε}-acetylated lysine. Preferably, the enzyme is HDAC1 (SEQ. ID. NO: 1), HDAC2 (SEQ. ID. NO: 2), HDAC3 (SEQ. ID. NO: 3), HDAC4 (SEQ. ID. NO: 4), HDAC5 (SEQ. ID. NO: 5), HDAC6 (SEQ. ID. NO: 6), HDAC7 (SEQ. ID. NO: 7), HDAC8 (SEQ. ID. NO: 8), HDAC9 (SEQ. ID. NO: 9), HDAC10 (SEQ. ID. NO: 10), or HDAC11 (SEQ. ID. NO: 11), any protein with 95% or greater sequence sirmilarity any of the said enzymes, or any fragment of any of the enzymes that retains catalytic deacetylase activity. Preferably, the enzyme is SIRT1 (SEQ. ID. NO: 12), SIRT2 (SEQ. ID. NO: 13), SIRT3 (SEQ. ID. NO: 14), SIRT4 (SEQ. ID. NO: 15), SIRT5 (SEQ. ID. NO: 16), SIRT6 (SEQ. ID. NO: 17), or SIRT7 (SEQ. ID. NO: 18), any protein with 95% or greater sequence similarity to any of the said enzymes, or any fragment of any of the enzymes that retains catalytic deacetylase activity. Most preferably, the enzyme is HDAC1.

Preferably, the protease is a member of the trypsin family of proteases. More preferably, the protease is trypsin or thrombin.

Preferably, the optical signal arises from a fluorescent moiety, or optical absorption, or from a fluorescence resonance energy transfer.

Preferably, polypeptide is less than or equal to 8 or 20 amino acids in length.

More preferably, the polypeptide is acetyl-Gly-Ala-(N^{ε}-acetyllysine)-AMC or (2-aminobenzoyl)-Gly-Ala-(N^{ε}-acetyllysine)-Ala-Ala-(3-(2,4-dinitrophenyl)-2,3-diaminopropionamide). Even more preferably acetyl-Gly-Ala-(N^{ε}-acetyllysine)-AMC.

In a third aspect, this invention is directed to a continuous method for measuring the inhibitory properties of a test compound towards the activity of an enzyme that catalyzes (a) the addition of an acetyl group to a residue capable of being acetylated or (b) removal of an acetyl group from an acetylated residue which method comprises incubating said enzyme with:

(i) a protease;

(ii) a polypeptide comprising:

(a) a recognition site for the protease;

(b) a residue, in which the acetylation state of the residue modifies the rate of cleavage of the polypeptide by the protease; and

(c) at least one chemical moiety, attached to the polypeptide, that possesses an optical signal that changes upon cleavage of the polypeptide;

in the presence of the test compound; and

(iii) measuring the rate of increase in the optical signal wherein the difference between the rate of increase of the optical signal in the presence of the test compound and the rate of increase of the optical signal in the absence of the test compound is indicative of inhibitory properties of the test compound.

Preferably, this invention is directed to a continuous method for measuring the inhibitory properties of a test compound towards an enzyme that catalyzes the addition or removal of acetyl group from N^{ε}-acetylated lysine residue which method comprises incubating said enzyme with:

(i) a protease;

(ii) a polypeptide comprising:

(a) a recognition site for the protease;

(b) a lysine or acetyllysine residue, in which the acetylation state of the lysine residue modifies the rate of cleavage of the polypeptide by the protease; and

(c) at least one chemical moiety, attached to the polypeptide, that possesses an optical signal that changes upon cleavage of the polypeptide; in the presence of the test compound; and

(iii) measuring the rate of increase in the optical signal wherein the difference between the rate of increase of the optical signal in the presence of the test compound and the rate of increase of the optical signal in the absence of the test compound is indicative of inhibitory properties of the test compound.

More preferably, the invention is directed to a continuous method for measuring the inhibitory properties of a test compound towards a histone deacetylase enzyme which method comprises incubating the histone deacetylase enzyme with:

(i) trypsin;

(ii) acetyl-Gly-Ala-(N^{ε}-acetyllysine)-AMC; in the presence and absence of the test compound; and

(iii) measuring the increase in fluorescence at 460 nm over time, using an excitation wavelength of 355 nm wherein the difference in the rate of increase of the fluorescence in the presence and absence of the test compound is indicative of inhibitory properties of the test compound.

Even more preferably, the histone deacetylase enzyme is HDAC1.

Particularly preferably, HDAC1 is incubated with the test compound for at least 5 minutes prior to addition of trypsin and acetyl-Gly-Ala-(N^{ε}-acetyllysine)-AMC.

### Definitions

Unless otherwise stated, the following terms used in the specification and claims are defined for the purposes of this Application and have the following meanings. Other terms used in the specification and claims have meanings recognized in the art.

The term “a” as used herein means at least one.

A “polypeptide” or “peptide” as used herein is a sequence of amino acids joined through amide bonds. The amino acids may be naturally occurring or non-natural. It is known in the art that side chains of several naturally occurring amino acids may be modified by the addition of chemical functionalities comprising methyl, acetyl, or phosphate groups.

A “moiety” as used herein is a molecule or portion of a molecule that possesses an optical signal or imparts an optical property to the molecule to which it is bound. For example, the amide form of 7-amino-4-methylcoumarin (AMC) when bound to a polypeptide has a weak fluorescence with an emission maximum at 395 nm. When the amide bond is cleaved, the free 7-amino-4-methylcoumarin has a very high fluorescence intensity with an emission maximum at 460 nm. Similarly, the p-nitroaniline moiety in its amide form is colorless, but when cleaved from the polypeptide it acquires an intense yellow color.

A “continuous” assay or method as used herein is one in which the process can be monitored on a constant basis without changing the process as a result of the measurement. This includes any technique in which the sample is monitored multiple times over the course of the reaction, but the preferred definition is one in which formation of a detectable product is directly related to the activity of the enzyme and the product can be quantitated in situ without any additional liquid handling or chemical reaction steps. For example, in the HDAC assay involving acetyl-Gly-Ala-(N^{ε}-acetyllysine)-AMC, the rate of production of free AMC is a measure of the rate of the deacetylase reaction. The production of free AMC can be monitored constantly or at arbitrarily small time intervals on the basis of its fluorescence, and thus the assay method is continuous. The reaction does not have to be stopped to detect a signal, and there are no extraction or purification steps necessary to isolate and quantitate the products.

A “proteolytic enzyme” or “protease” as used herein is an enzyme that catalyzes the cleavage of amide bonds within a polypeptide. Trypsin and thrombin are examples of proteases.

An “optical signal” as used herein is any response to illumination of a moiety that can be used to detect or quantitate the given moiety. Absorption and fluorescence are examples of optical signals.

A “residue” as used herein in the context of a polypeptide is an amino acid side chain that occurs within the polypeptide chain. The residue can be naturally or non-naturally occurring. For example, hydroxymethyl, thiomethyl, are naturally occurring residues.

A “recognition site” as used herein is a sequence of amino acids within a polypeptide that allows a protease enzyme to bind to and cleave the said polypeptide.

Unless otherwise stated, “acetyllysine” as used herein refers to lysine acetylated at the ε-amino nitrogen.

“Inhibition” as used herein is a decrease in the rate of an enzyme-catalyzed reaction as a result of a compound binding to the enzyme and disrupting the interaction of the enzyme with its substrate. For the assays described herein, inhibition of a deacetylase enzyme will result in a decrease in the magnitude of the optical signal as compared with a reaction in the absence of an inhibitor.

### EXAMPLES

### Example 1

### Synthesis of acetyl-Gly-Ala-(N-acetyl-Lys)-AMC

tert-Boc (N-Acetyl-Lys)-AMC (445 mg, 1 mmol, purchased from Bachem) was dissolved in 4 M HCL in dioxane to provide H-(N-acetyl-Lys)-AMC as a white solid. To a solution of H-(N-acetyl-Lys)-AMC in DMF (5 ml) was added Ac-Gly-Ala-OH (188 mg, 1 mmol) using PyBOP (520 mg, 1 mmol), HOBt (135 mg, 1 mmol), and NMM (0.296 ml, 2 mmol). The reaction mixture was stirred for 1 h and monitored by MS/LC for the presence of H-(N-acetyl-Lys)-AMC. Additional amounts of PyBOP (260 mg, 0.5 mmol), HOBt (70 mg, 0.5 mmol), and NMM (0.146 ml, 1 mmol) was added and the stirring was continued for additional 4 h after which the product was isolated in quantative yield.

### Example 2

Measurement of Histone Deacetylase Activity Using a FRET Substrate

HDAC8 was cloned, isolated, and purified as described in the literature (Buggy, et al. Biochem. J. 2000, 350, 199-205). The peptide 2-aminobenzoyl-Gly-Ala-(N^{ε}-acetyllysine)-Ala-Ala-(3-dinitrophenyl-(L)-2,3-diaminopropionamide) (peptide 2) was purchased from California Peptide Research, Inc. The measurement was performed in a reaction volume of 100 μL using a 96-well assay plate. HDAC8 (approx. 400 nM final concentration) in 50 mM HEPES, 100 mM KCl, 0.001% Tween-20, 0.01% bovine serum albumin, 5% DMSO, pH 7.4, was mixed with bovine trypsin (Sigma, 50 nM final concentration) and peptide 2 (20 μM final concentration). The reaction was monitored for 1 hour in a fluorescence plate reader, using an excitation wavelength of 320 nm and a detection wavelength of 405 nm. An increase of fluorescence with time was used as the measure of reaction rate.

### Example 3

### Determination of the Inhibitory Properties of Chemical Compounds

Measurements were performed in a reaction volume of 100 μL using 96-well assay plates. HDAC-1 (200 pM final concentration) in reaction buffer (50 mM HEPES, 100 mM KCl, 0.001% Tween-20, 5% DMSO, pH 7.4) was mixed with inhibitor at various concentrations and allowed to incubate for 30 minutes, after which trypsin and acetyl-Gly-Ala-(N-acetyl-Lys)-AMC were added to final concentrations of 50 nM and 25 μM, respectively, to initiate the reaction. Negative control reactions were performed in the absence of inhibitor in replicates of eight.

The reactions were monitored in a fluorescence plate reader. After a 30 minute lag time, the fluorescence was measured over a 30 minute time frame using an excitation wavelength of 355 nm and a detection wavelength of 460 nm. The increase in fluorescence with time was used as the measure of the reaction rate. Inhibition constants were obtained using the program BatchKi (Kuzmic et al. Anal. Biochem. 2000, 286, 45-50).

### Sequence Identification

Sequence IDs utiltize the accession codes of the NCBI Reference Sequence project at the National Institutes of Health. Supporting literature references are included.

SEQ ID. NO: 1 (Taunton, et al. Science 1996, 272, 408-411; RefSeq NP_{—}004955)

SEQ ID. NO: 2 (Yang, et al. Proc. Natl. Acad. Sci. U.S.A. 1996, 93, 12845-12850; RefSeq NP_{—}001518)

SEQ ID. NO: 3 (Yang et al. J. Biol. Chem. 1997, 272, 28001-28007; RefSeq NP_{—}003874)

SEQ ID. NO: 4 (Grozinger, et al. Proc. Natl. Acad. Sci. U.S.A. 1999, 96, 4868-4873; RefSeq NP_{—}006028)

SEQ ID. NO: 5 (Grozinger, et al. Proc. Natl. Acad. Sci. U.S.A. 1999, 96, 4868-4873; RefSeq NP_{—}631944)

SEQ ID NO: 6 (Grozinger, et al. Proc. Natl. Acad. Sci. U.S.A. 1999, 96, 4868-4873; RefSeq NP_{—}006035)

SEQ ID. NO: 7 (Kao, et al. Genes Dev. 2000, 14, 55-66; RefSeq NP_{—}056216)

SEQ ID. NO: 8 (Buggy, et al. Biochem. J. 2000, 350, 199-205; RefSeq NP_{—}060956)

SEQ ID. NO: 9 (Zhou, et al. Proc. Natl. Acad. Sci. U.S.A. 2001, 98, 10572-10577; RefSeq NP_{—}478056)

SEQ ID. NO: 10 (Kao, et al. J. Biol. Chem. 2002, 277, 187-193; RefSeq NP_{—}114408)

SEQ ID. NO: 11 (Gao, et al. J. Biol. Chem. 2002, 277, 25748-25755; RefSeq NP_{—}079103)

SEQ ID. NO: 12 (Frye, Biochem. Biophys. Res. Commun. 1999, 260, 273-279; RefSeq NP_{—}036370)

SEQ ID. NO: 13 (Frye, Biochem. Biophys. Res. Commun. 1999, 260, 273-279; RefSeq NP_{—}036369)

SEQ ID. NO: 14 (Frye, Biochem. Biophys. Res. Commun. 1999, 260, 273-279; RefSeq NP_{—}036371)

SEQ ID. NO: 15 (Frye, Biochem. Biophys. Res. Commun. 1999, 260, 273-279; RefSeq NP_{—}036372)

SEQ ID. NO: 16 (Frye, Biochem. Biophys. Res. Commun. 1999, 260, 273-279; RefSeq NP_{—}036373)

SEQ ID. NO: 17 (Frye, Biochem. Biophys. Res. Commun. 2000, 273, 793-798; RefSeq NP_{—}057623)

SEQ ID NO: 18 (Frye, Biochem. Biophys. Res. Commun. 2000, 273, 793-798; RefSeq NP_{—}057622)

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

What is claimed is:

1. A continuous method for measuring the activity of an enzyme that catalyzes (a) the addition of an acetyl group to a residue capable of being acetylated or (b) removal of an acetyl group from an acetylated residue which method comprises incubating said enzyme with:
(i) a protease;
(ii) a polypeptide comprising:
(a) a recognition site for the protease;
(b) a residue, in which the acetylation state of the residue modifies the rate of cleavage of the polypeptide by the protease; and
(c) at least one chemical moiety, attached to the polypeptide, that possesses an optical signal that changes upon cleavage of the polypeptide; and
(iii) measuring the change in the optical signal.

2. A continuous method for measuring the activity of an enzyme that catalyzes the addition of an acetyl group to a lysine residue or an enzyme that catalyzes the removal of acetyl group from N^{ε}-acetylated lysine residue which method comprises incubating said enzyme with:
(i) a protease;
(ii) a polypeptide comprising:
(a) a recognition site for the protease;
(b) a lysine or acetyllysine residue, in which the acetylation state of the residue modifies the rate of cleavage of the polypeptide by the protease; and
(c) at least one chemical moiety, attached to the polypeptide, that possesses an optical signal that changes upon cleavage of the polypeptide; and
(iii) measuring the change in the optical signal.

3. A continuous method for measuring the activity of an enzyme that catalyzes the removal of acetyl group from N^{ε}-acetylated lysine residue which method comprises incubating said enzyme with:
(i) a protease;
(ii) a polypeptide comprising:
(a) a recognition site for the protease;
(b) an acetyllysine residue, which residue when acetylated attenuates the rate of cleavage of the polypeptide by the protease; and
(c) at least one chemical moiety, attached to the polypeptide, that possesses an optical signal that changes upon cleavage of the polypeptide; and
(iii) measuring the change in the optical signal.

4. The method of claim 3 where the enzyme is HDAC1 (SEQ. ID. NO: 1), HDAC2 (SEQ. ID. NO: 2), HDAC3 (SEQ. ID. NO: 3), HDAC4 (SEQ. ID. NO: 4), HDAC5 (SEQ. ID. NO: 5), HDAC6 (SEQ. ID. NO: 6), HDAC7 (SEQ. ID. NO: 7), HDAC8 (SEQ. ID. NO: 8), HDAC9 (SEQ. ID. NO: 9), HDAC10 (SEQ. ID. NO: 10), or HDAC11 (SEQ. ID. NO: 11), any protein with 95% or greater sequence similarity any of the said enzymes, or any fragment of any of the enzymes that retains catalytic deacetylase activity.

5. The method of claim 3 where the enzyme is SIRT1 (SEQ. ID. NO: 12), SIRT2 (SEQ. ID. NO: 13), SIRT3 (SEQ. ID. NO: 14), SIRT4 (SEQ. ID. NO: 15), SIRT5 (SEQ. ID. NO: 16), SIRT6 (SEQ. ID. NO: 17), or SIRT7 (SEQ. ID. NO: 18), any protein with 95% or greater sequence similarity to any of the said enzymes, or any fragment of any of the enzymes that retains catalytic deacetylase activity.

6. The method of claim 3 where the protease is a member of the trypsin family of proteases.

7. The method of claim 3 where the protease is trypsin or thrombin.

8. The method of claim 3 where the optical signal arises from a fluorescent moiety.

9. The method of claim 3 where the optical signal arises from optical absorption.

10. The method of claim 3 where the optical signal arises from a fluorescence resonance energy transfer.

11. The method of claim 7 where the polypeptide is acetyl-Gly-Ala-(N^{ε}-acetyllysine)-AMC.

12. The method of claim 10 where the polypeptide is (2-aminobenzoyl)-Gly-Ala-(N^{ε}-acetyllysine)-Ala-Ala-(3-(2,4-dinitrophenyl)-2,3-diaminopropionamide).

13. The method of claim 3 where the polypeptide is less than or equal to 20 amino acids in length.

14. The method of claim 3 where the polypeptide is less than or equal to 8 amino acids in length.

15. A continuous method for measuring the activity of a histone deacetylase enzyme which method comprises incubating the histone deacetylase enzyme with:
(i) trypsin;
(ii) acetyl-Gly-Ala-(N^{ε}-acetyllysine)-AMC; and
(iii) measuring the increase in fluorescence at 460 nm over time, using an excitation wavelength of 355 nm.

16. The method of claim 15 where the histone deacetylase enzyme is HDAC1.

17. A continuous method for measuring the inhibitory properties of a test compound towards an enzyme that catalyzes the addition or removal of acetyl group from N^{ε}- acetylated lysine residue which method comprises incubating said enzyme with:
(i) a protease;
(ii) a polypeptide comprising:
(a) a recognition site for the protease;
(b) a lysine or acetyllysine residue, in which the acetylation state of the lysine residue modifies the rate of cleavage of the polypeptide by the protease; and
(c) at least one chemical moiety, attached to the polypeptide, that possesses an optical signal that changes upon cleavage of the polypeptide; in the presence of the test compound;
(iii) measuring the rate of increase in the optical signal wherein the difference between the rate of increase of the optical signal in the presence of the test compound and the rate of increase of the optical signal in the absence of the test compound is indicative of inhibitory properties of the test compound.

18. A continuous method for measuring the inhibitory properties of a test compound towards a histone deacetylase enzyme which method comprises incubating the histone deacetylase enzyme with:
(i) trypsin;
(ii) acetyl-Gly-Ala-(N^{ε}-acetyllysine)-AMC; in the presence and absence of the test compound; and
(iii) measuring the increase in fluorescence at 460 nm over time, using an excitation wavelength of 355 nm wherein the difference in the rate of increase of the fluorescence in the presence and absence of the test compound is indicative of inhibitory properties of the test compound.

19. The method of claim 18 where the histone deacetylase enzyme is HDAC1.

20. The method of claim 19 wherein HDAC1 is incubated with the test compound for at least 5 minutes prior to addition of trypsin and acetyl-Gly-Ala-(N^{ε}-acetyllysine)-AMC.


SEQUENCE LISTING (18 sequences)

SEQ ID NO: 1 - protein, 482 aa, Homo sapiens
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 2 - protein, 488 aa, Homo sapiens
MAYSQGGGKKKVCYYYDGDIGNYYYGQGHPMKPHRIRMTHNLLLNYGLYRKMEIYRPHKATAEEMTKYHSDEYIKFLRSIRPDNMSEYSKQMHIFNVGEDCPAFDGLFEFCQLSTGGSVAGAVKLNRQQTDMAVNWAGGLHHAKKYEASGFCYVNDIVLAILELLKYHQRVLYIDIDIHHGDGVEEAFYTTDRVMTVSFHKYGEYFPGTGDLRDIGAGKGKYYAVNFPMCDGIDDESYGQIFKPIISKVMEMYQPSAVVLQCGADSLSGDRLGCFNLTVKGHAKCVEVVKTFNLPLLMLGGGGYTIRNVARCWTYETAVALDCEIPNELPYNDYFEYFGPDFKLHISPSNMTNQNTPEYMEKIKQRLFENLRMLPHAPGVQMQAIPEDAVHEDSGDEDGEDPDKRISIRASDKRIACDEEFSDSEDEGEGGRRNVADHKKGAKKARIEEDKKETEDKKTDVKEEDKSKDNSGEKTDTKGTKSEQLSNP

SEQ ID NO: 3 - protein, 428 aa, Homo sapiens
MAKTVAYFYDPDVGNFHYGAGHPMKPHRLALTHSLVLHYGLYKKMIVFKPYQASQHDMCRFHSEDYIDFLQRVSPTNMQGFTKSLNAFNVGDDCPVFPGLFEFCSRYTGASLQGATQLNNKICDIAINWAGGLHHAKKFEASGFCYVNDIVIGILELLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQIRQTIFENLKMLNHAPSVQIHDVPADLLTYDRTDEADAEERGPEENYSRPEAPNEFYDGDHDNDKESDVEI

SEQ ID NO: 4 - protein, 1084 aa, Homo sapiens
MSSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 5 - protein, 1037 aa, Homo sapiens
MNSPNESDGMSGREPSLEILPRTSLHSIPVTVEVKPVLPRAMPSSMGGGGGGSPSPVELRGALVGSVDPTLREQQLQQELLALKQQQQLQKQLLFAEFQKQHDHLTRQHEVQLQKHLKQQQEMLAAKQQQEMLAAKRQQELEQQRQREQQRQEELEKQRLEQQLLILRNKEKSKESAIASTEVKLRLQEFLLSKSKEPTPGGLNHSLPQHPKCWGAHHASLDQSSPPQSGPPGTPPSYKLPLPGPYDSRDDFPLRKTASEPNLKVRSRLKQKVAERRSSPLLRRKDGTVISTFKKRAVEITGAGPGASSVCNSAPGSGPSSPNSSHSTIAENGFTGSVPNIPTEMLPQHRALPLDSSPNQFSLYTSPSLPNISLGLQATVTVTNSHLTASPKLSTQQEAERQALQSLRQGGTLTGKFMSTSSIPGCLLGVALEGDGSPHGHASLLQHVLLLEQARQQSTLIAVPLHGQSPLVTGERVATSMRTVGKLPRHRPLSRTQSSPLPQSPQALQQLVMQQQHQQFLEKQKQQQLQLGKILTKTGELPRQPTTHPEETEEELTEQQEVLLGEGALTMPREGSTESESTQEDLEEEDEEDDGEEEEDCIQVKDEEGESGAEEGPDLEEPGAGYKKLFSDAQPLQPLQVYQAPLSLATVPHQALGRTQSSPAAPGGMKSPPDQPVKHLFTTGPISQKMYAVLPCGGIGVDSDTVWNEMHSSSAVRMAVGCLLELAFKVAAGELKNGFAIIRPPGHHAEESTAMGFCFFNSVAITAKLLQQKLNVGKVLIVDWDIHHGNGTQQAFYNDPSVLYISLHRYDNGNFFPGSGAPEEVGGGPGVGYNVNVAWTGGVDPPIGDVEYLTAFRTVVMPIAHEFSPDVVLVSAGFDAVEGHLSPLGGYSVTARCFGHLTRQLMTLAGGRVVLALEGGHDLTAICDASEACVSALLSVELQPLDEAVLQQKPNINAVATLEKVIEIQSKHWSCVQKFAAGLGRSLREAQAGETEEAETVSAMALLSVGAEQAQAAAAREHSPRPAEEPMEQEPAL

SEQ ID NO: 6 - protein, 1215 aa, Homo sapiens
MTSTGQDSTTTRQRRSRQNPQSPPQDSSVTSKRNIKKGAVPRSIPNLAEVKKKGKMKKLGQAMEEDLIVGLQGMDLNLEAEALAGTGLVLDEQLNEFHCLWDDSFPEGPERLHAIKEQLIQEGLLDRCVSFQARFAEKEELMLVHSLEYIDLMETTQYMNEGELRVLADTYDSVYLHPNSYSCACLASGSVLRLVDAVLGAEIRNGMAIIRPPGHHAQHSLMDGYCMFNHVAVAARYAQQKHRIRRVLIVDWDVHHGQGTQFTFDQDPSVLYFSIHRYEQGRFWPHLKASNWSTTGFGQGQGYTINVPWNQVGMRDADYIAAFLHVLLPVALEFQPQLVLVAAGFDALQGDPKGEMAATPAGFAQLTHLLMGLAGGKLILSLEGGYNLRALAEGVSASLHTLLGDPCPMLESPGAPCRSAQASVSCALEALEPFWEVLVRSTETVERDNMEEDNVEESEEEGPWEPPVLPILTWPVLQSRTGLVYDQNMMNHCNLWDSHHPEVPQRILRIMCRLEELGLAGRCLTLTPRPATEAELLTCHSAEYVGHLRATEKMKTRELHRESSNFDSIYICPSTFACAQLATGAACRLVEAVLSGEVLNGAAVVRPPGHHAEQDAACGFCFFNSVAVAARHAQTISGHALRILIVDWDVHHGNGTQHMFEDDPSVLYVSLHRYDHGTFFPMGDEGASSQIGRAAGTGFTVNVAWNGPRMGDADYLAAWHRLVLPIAYEFNPELVLVSAGFDAARGDPLGGCQVSPEGYAHLTHLLMGLASGRIILILEGGYNLTSISESMAACTRSLLGDPPPLLTLPRPPLSGALASITETIQVHRRYWRSLRVMKVEDREGPSSSKLVTKKAPQPAKPRLAERMTTREKKVLEAGMGKVTSASFGEESTPGQTNSETAVVALTQDQPSEAATGGATLAQTISEAAIGGAMLGQTTSEEAVGGATPDQTTSEETVGGAILDQTTSEDAVGGATLGQTTSEEAVGGATLAQTTSEAAMEGATLDQTTSEEAPGGTELIQTPLASSTDHQTPPTSPVQGTTPQISPSTLIGSLRTLELGSESQGASESQAPGEENLLGEAAGGQDMADSMLMQGSRGLTDQAIFYAVTPLPWCPHLVAVCPIPAAGLDVTQPCGDCGTIQENWVCLSCYQVYCGRYINGHMLQHHGNSGHPLVLSYIDLSAWCYYCQAYVHHQALLDVKNIAHQNKFGEDMPHPH

SEQ ID NO: 7 - protein, 952 aa, Homo sapiens
MDLRVGQRPPVEPPPEPTLLALQRPQRLHHHLFLAGLQQQRSVEPMRLSMDTPMPELQVGPQEQELRQLLHKDKSKRSAVASSVVKQKLAEVILKKQQAALERTVHPNSPGIPYRTLEPLETEGATRSMLSSFLPPVPSLPSDPPEHFPLRKTVSEPNLKLRYKPKKSLERRKNPLLRKESAPPSLRRRPAETLGDSSPSSSSTPASGCSSPNDSEHGPNPILGSEALLGQRLRLQETSVAPFALPTVSLLPAITLGLPAPARADSDRRTHPTLGPRGPILGSPHTPLFLPHGLEPEAGGTLPSRLQPILLLDPSGSHAPLLTVPGLGPLPFHFAQSLMTTERLSGSGLHWPLSRTRSEPLPPSATAPPPPGPMQPRLEQLKTHVQVIKRSAKPSEKPRLRQIPSAEDLETDGGGPGQVVDDGLEHRELGHGQPEARGPAPLQQHPQVLLWEQQRLAGRLPRGSTGDTVLLPLAQGGHRPLSRAQSSPAAPASLSAPEPASQARVLSSSETPARTLPFTTGLIYDSVMLKHQCSCGDNSRHPEHAGRIQSIWSRLQERGLRSQCECLRGRKASLEELQSVHSERHVLLYGTNPLSRLKLDNGKLAGLLAQRMFVMLPCGGVGVDTDTIWNELHSSNAARWAAGSVTDLAFKVASRELKNGFAVVRPPGHHADHSTAMGFCFFNSVAIACRQLQQQSKASKILIVDWDVHHGNGTQQTFYQDPSVLYISLHRHDDGNFFPGSGAVDEVGAGSGEGFNVNVAWAGGLDPPMGDPEYLAAFRIVVMPIAREFSPDLVLVSAGFDAAEGHPAPLGGYHVSAKCFGYMTQQLMNLAGGAVVLALEGGHDLTAICDASEACVAALLGNRVDPLSEEGWKQKPNLNAIRSLEAVIRVHSKYWGCMQRLASCPDSWVPRVPGADKEEVEAVTALASLSVGILAEDRPSEQLVEEEEPMNL

SEQ ID NO: 8 - protein, 377 aa, Homo sapiens
MEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVV

SEQ ID NO: 9 - protein, 1011 aa, Homo sapeins
MHSMISSVDVKSEVPVGLEPISPLDLRTDLRMMMPVVDPVVREKQLQQELLLIQQQQQIQKQLLIAEFQKQHENLTRQHQAQLQEHIKELLAIKQQQELLEKEQKLEQQRQEQEVERHRREQQLPPLRGKDRGRERAVASTEVKQKLQEFLLSKSATKDTPTNGKNHSVSRHPKLWYTAAHHTSLDQSSPPLSGTSPSYKYTLPGAQDAKDDFPLRKTASEPNLKVRSRLKQKVAERRSSPLLRRKDGNVVTSFKKRMFEVTESSVSSSSPGSGPSSPNNGPTGSVTENETSVLPPTPHAEQMVSQQRILIHEDSMNLLSLYTSPSLPNITLGLPAVPSQLNASNSLKEKQKCETQTLRQGVPLPGQYGGSIPASSSHPHVTLEGKPPNSSHQALLQHLLLKEQMRQQKLLVAGGVPLHPQSPLATKERISPGIRGTHKLPRHRPLNRTQSAPLPQSTLAQLVIQQQHQQFLEKQKQYQQQIHMNKLLSKSIEQLKQPGSHLEEAEEELQGDQAMQEDRAPSSGNSTRSDSSACVDDTLGQVGAVKVKEEPVDSDEDAQIQEMESGEQAAFMQQPFLEPTHTRALSVRQAPLAAVGMDGLEKHRLVSRTHSSPAASVLPHPAMDRPLQPGSATGIAYDPLMLKHQCVCGNSTTHPEHAGRIQSIWSRLQETGLLNKCERIQGRKASLEEIQLVHSEHHSLLYGTNPLDGQKLDPRILLGDDSQKFFSSLPCGGLGVDSDTIWNELHSSGAARMAVGCVIELASKVASGELKNGFAVVRPPGHHAEESTAMGFCFFNSVAITAKYLRDQLNISKILIVDLDVHHGNGTQQAFYADPSILYISLHRYDEGNFFPGSGAPNEVGTGLGEGYNINIAWTGGLDPPMGDVEYLEAFRTIVKPVAKEFDPDMVLVSAGFDALEGHTPPLGGYKVTAKCFGHLTKQLMTLADGRVVLALEGGHDLTAICDASEACVNALLGNELEPLAEDILHQSPNMNAVISLQKIIEIQSMSLKFS

SEQ ID NO: 10 - protein, 669 aa, Homo sapiens
MGTALVYHEDMTATRLLWDDPECEIERPERLTAALDRLRQRGLEQRCLRLSAREASEEELGLVHSPEYVSLVRETQVLGKEELQALSGQFDAIYFHPSTFHCARLAAGAGLQLVDAVLTGAVQNGLALVRPPGHHGQRAAANGFCVFNNVAIAAAHAKQKHGLHRILVVDWDVHHGQGIQYLFEDDPSVLYFSWHRYEHGRFWPFLRESDADAVGRGQGLGFTVNLPWNQVGMGNADYVAAFLHLLLPLAFEFDPELVLVSAGFDSAIGDPEGQMQATPECFAHLTQLLQVLAGGRVCAVLEGGYHLESLAESVCMTVQTLLGDPAPPLSGPMAPCQSALESIQSARAAQAPHWKSLQQQDVTAVPMSPSSHSPEGRPPPLLPGGPVCKAAASAPSSLLDQPCLCPAPSVRTAVALTTPDITLVLPPDVIQQEASALREETEAWARPHESLAREEALTALGKLLYLLDGMLDGQVNSGIAATPASAAAATLDVAVRRGLSHGAQRLLCVALGQLDRPPDLAHDGRSLWLNIRGKEAAALSMFHVSTPLPVMTGGFLSCILGLVLPLAYGFQPDLVLVALGPGHGLQGPHAALLAAMLRGLAGGRVLALLEENSTPQLAGILARVLNGEAPPSLGPSSVASPEDVQALMYLRGQLEPQWKMLQCHPHLVA

SEQ ID NO: 11 - protein, 347 aa, Homo sapiens
MLHTTQLYQHVPETPWPIVYSPRYNITFMGLEKLHPFDAGKWGKVINFLKEEKLLSDSMLVEAREASEEDLLVVHTRRYLNELKWSFAVATITEIPPVIFLPNFLVQRKVLRPLRTQTGGTIMAGKLAVERGWAINVGGGFHHCSSDRGGGFCAYADITLAIKFLFERVEGISRATIIDLDAHQGNGHERDFMDDKRVYIMDVYNRHIYPGDRFAKQAIRRKVELEWGTEDDEYLDKVERNIKKSLQEHLPDVVVYNAGTDILEGDRLGGLSISPAGIVKRDELVFRMVRGRRVPILMVTSGGYQKRTARIIADSILNLFGLGLIGPESPSVSAQNSDTPLLPPAVP

SEQ ID NO: 12 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 13 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 14 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 15 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 16 - protein, 310 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 17 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMEHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 18 - protein, 400 aa, Homo sapiens
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT